(19) United States
(12) Patent Application Publication
(10) Pub. No.: US 2019/0002529 A1
(43) Pub. Date: Jan. 3, 2019

(54) Assembly of Intermediate Filament Proteins in to Filamentous Materials

(71) Applicant: The Government of the United States of America, as represented by the Secretary of the Navy, Arlington, VA (US)

(72) Inventor: Joshua M. Kogot, Panama City, FL (US)

(21) Appl. No.: 16/013,082

(22) Filed: Jun. 20, 2018

Related U.S. Application Data
(60) Provisional application No. 62/528,207, filed on Jul. 3, 2017.

(51) Int. Cl. C07K 14/78; C07K 1/14

(52) U.S. Cl. CPC C07K 14/78; C07K 1/14

(57) ABSTRACT

Intermediate filament (IF) recombinant protein, such as from hagfish, can be purified under denaturing conditions either as a single protein (alpha or gamma alone) or assembled in a 1:1 molar ratio (alpha plus gamma). Step-down renaturation (protein refolding) or rapid dilution refolding is followed by formation of IF threads.

### BACKGROUND

The hagfish animals (class Agnatha, order Cyclostomata) produce a defensive slime or mucus that is reinforced with keratin-like intermediate filament (IF) threads that have high tensile strength properties akin to spider silk. Hagfish slime filaments include two proteins, alpha and gamma, in a 1:1 ratio. The alpha and gamma proteins are keratin-like proteins that undergo an α⋅helix to β⋅sheet transition during stress or lengthening of the filaments, which lends to its impressive tensile and shear strength properties. The slime thread filaments can reform from the individual components of the natural hagfish slime material. Prior research with recombinant proteins (Fu, et al 2015) showed that the alpha and gamma proteins could be grown in E. coli and that were located in insoluble inclusion bodies. The proteins were purified by size exclusion chromatography, which is not amenable to large scale batch production or purification nor on-column refolding methods. The proteins were refolded over four days in a stepwise removal of denaturants. The recombinant hagfish proteins were self-assembled by centrifugal concentration in a 100 kDa membrane, which resulted in micro scale alpha-plus⋅gamma protein but no mention or evidence of formation of the macroscale, bulk filament threads.

A need exists for an improved process for preparing recombinant IF proteins.

### BRIEF DESCRIPTION OF THE DRAWINGS

FIG. 1 is an image of SDS-PAGE of recombinant, polyHis-tag alpha and gamma proteins of hagfish intermediate filaments.

FIG. 2 is a schematic illustration of an exemplary procedure for the preparation of intermediate filaments.

FIG. 3 is a schematic illustration of an exemplary procedure using native untagged intermediate filament proteins.

FIGS. 4A and 4B are scanning electron microscope (SEM) images showing a single synthetic hagfish filament.

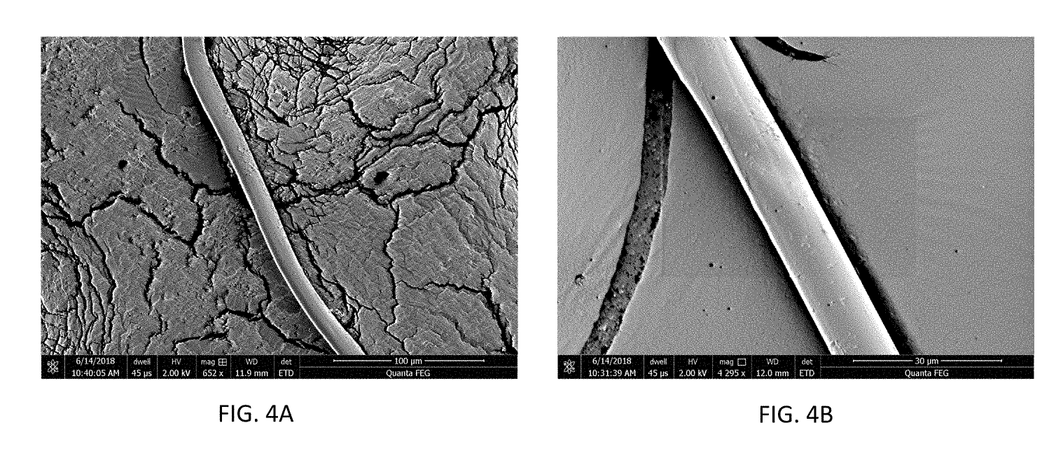

FIGS. 5A-5D are optical microscope image showing multiple, synthetic, single filament hagfish threads.

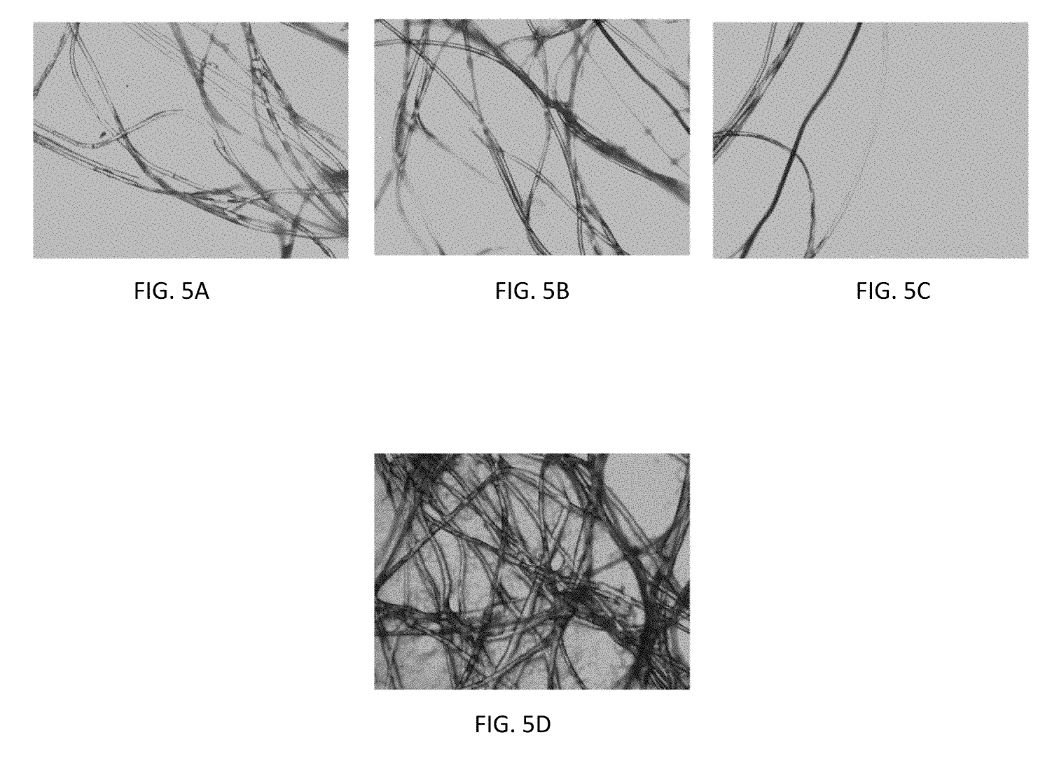

### DETAILED DESCRIPTION

### Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used herein, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

Overview

The invention relates to techniques for the rapid preparation, high purity process of recombinant intermediate filament proteins and thread assembly.

In one embodiment, a known hagfish protein amino acid sequence is used to create DNA sequence (optionally codon optimized for the expressing organism) incorporated into an expression vector. In various embodiments, the expression construct optionally incorporates a N-terminal or C-terminal polyhistidine-tag protein, (optionally with a cleavable His-tag), small molecule inducible promoter (such as lac operon), and/or antibiotic resistance for vector selection.

In further embodiments, a known hagfish protein amino acid sequence is used to create DNA sequence incorporated into an expression vector for native, untagged protein.

Such intermediate filament (IF) recombinant proteins from hagfish can be processed as a single protein (alpha or gamma alone) or assembled in a 1:1 molar ratio (alpha plus gamma) during step-down renaturation (protein refolding) or rapid dilution refolding.

In additional embodiments, a nucleic acid construct codes for the expression of both alpha and gamma hagfish intermediate filament proteins as a continuous single protein chain joined by a flexible linker, along with one or more an optionally cleavable polyhistidine tags.

Optionally, a nucleic acid construct codes for the expression of both alpha and gamma on a single expression vector with one or more small molecule inducible promoters.

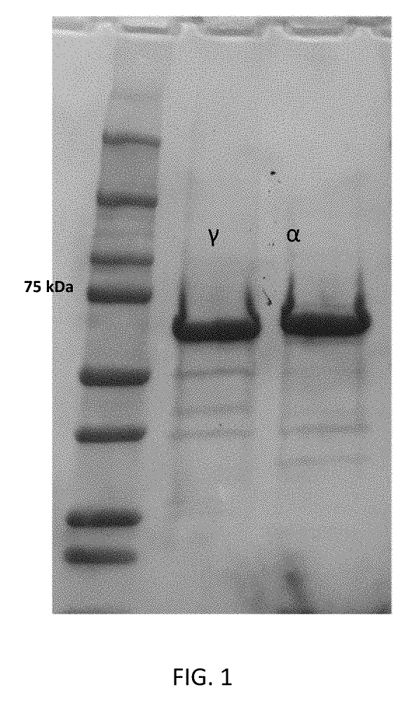

FIG. 1 shows results of SDS-PAGE of recombinant, polyHis-tag alpha and gamma proteins of hagfish intermediate filaments.

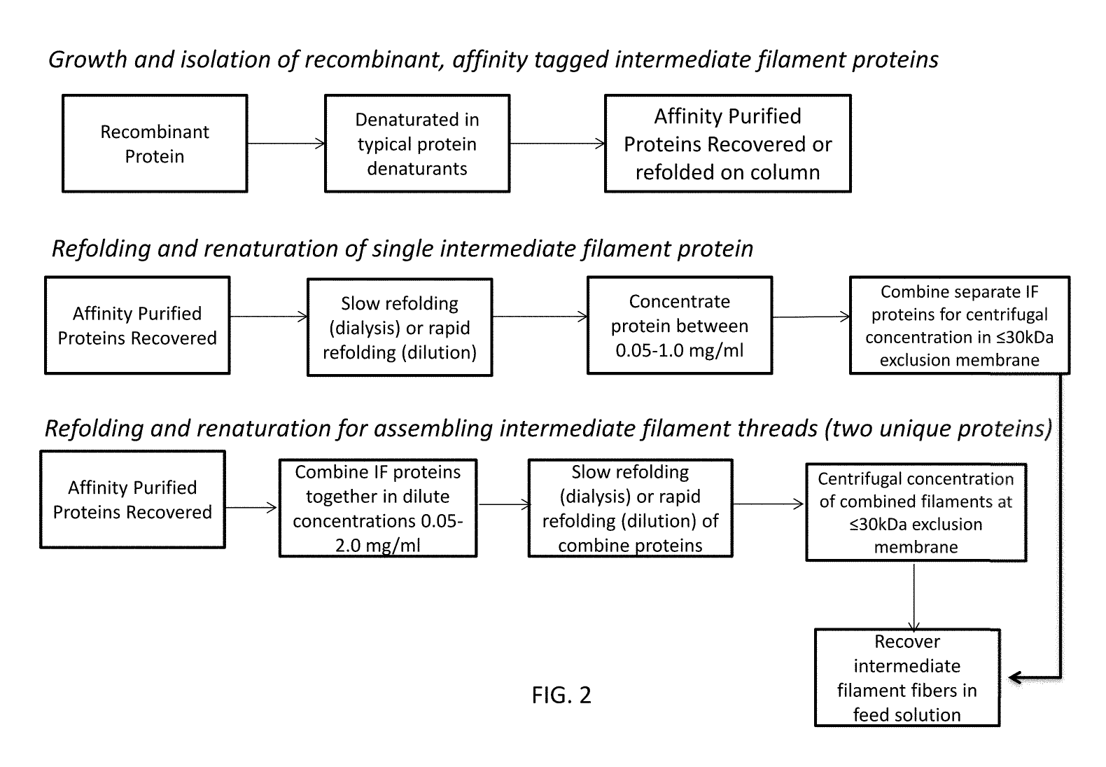

Referring the exemplary procedure shown in FIG. 2, the recombinant polyHis-tag or untagged intermediate filament protein is isolated in inclusion bodies during expression in bacterial host such as E. coli, lysed using standard chemical lysis methods (lysozyme, PMSF, deoxycholate), collected as an insoluble inclusion body (2), and redissolved in protein denaturant such as aqueous solutions of 0-8M Urea or 0-7M guanidinium hydrochloride (3), and affinity purified using immobilized metal affinity chromatography (IMAC) for high purity protein in one-step purification (4). The IMAC purification is amenable to high concentrations of denaturants without affecting purification and allows for on-column rapid refolding of the protein (5). The on-column refolding is an advantage of IMAC that improves the speed of refolding from 5 days to less than one day. The denatured, purified recombinant protein is subjected to either on-column protein refolding, renaturation during slow, step-down refolding. using decreasing denaturant concentrations (6), or is rapidly diluted in a suitable aqueous buffer for rapid protein refolding. The renaturation during slow, step-down denaturant concentrations or rapid dilution can be performed on each IF protein separately (alpha and gamma) (6) or in combination of the two-proteins (alpha plus gamma at 1:1) (7) during each refolding step. The IF thread is formed by combining the two protein components individually (8) or using the combined refolded IF proteins in a centrifugal concentration (9) step or other suitable concentration-self-assembly process using an exclusion membrane no greater than 30 kDa with an aqueous buffer containing magnesium or calcium ions. The IF thread is visualized in the feed or supernatant during concentration and is manually removed (10).

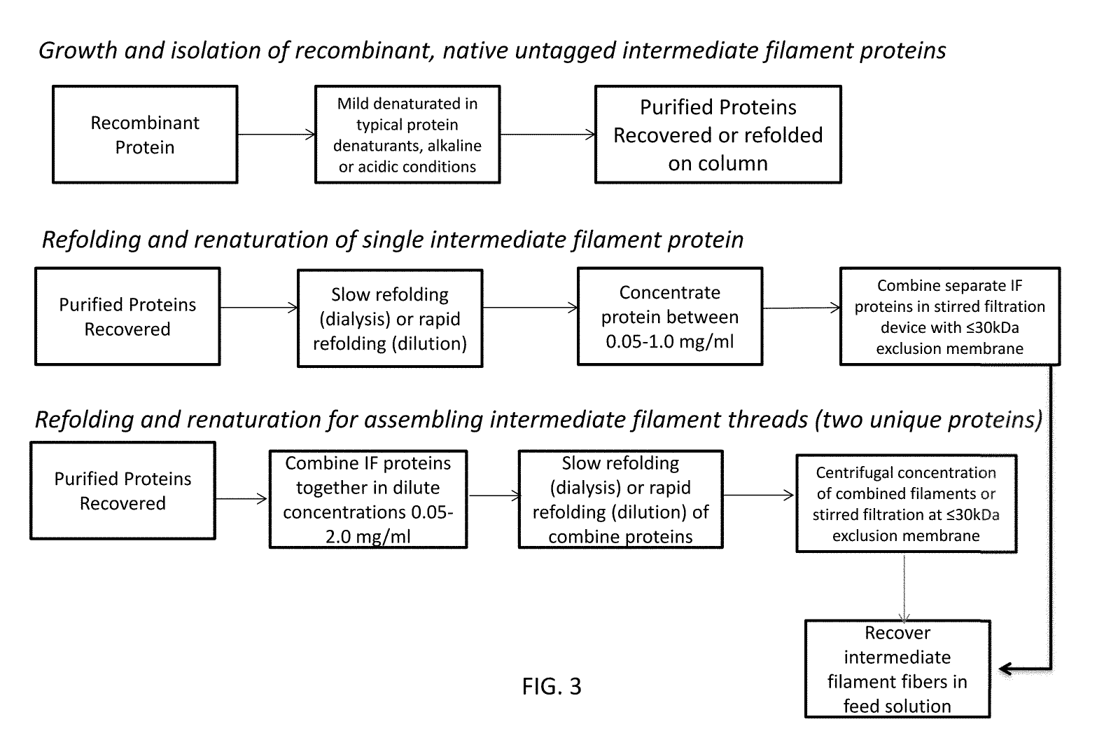

Referring the alternative procedure shown in FIG. 3, the recombinant polyHis-tag or untagged intermediate filament protein is isolated in inclusion bodies during expression in bacterial host such as E. coli, lysed using standard chemical lysis methods (lysozyme, PMSF, deoxycholate), collected as an insoluble inclusion body (2), and redissolved in a minimal concentration of protein denaturant such as aqueous solutions of 0-4M Urea or 0-4M guanidinium hydrochloride, acidic pH<7 or alkaline pH>7 solutions, with or without detergents, salts, or (3), and purified using ion exchange, hydrophobic interaction, or size exclusion chromatography for high purity protein in one-step purification (4). The purification method is to on-column rapid refolding of the protein (5). The on-column refolding is an advantage to improve the speed of refolding from 5 days to less than one day. The denatured, purified recombinant protein is subjected to either on-column protein refolding, renaturation during slow, step-down refolding. using decreasing denaturant concentrations (6), or is rapidly diluted in a suitable aqueous buffer for rapid protein refolding. The renaturation during slow, step-down denaturant concentrations or rapid dilution can be performed on each IF protein separately (alpha and gamma) (6) or in combination of the two-proteins (alpha plus gamma at 1:1) (7) during each refolding step. The IF thread is formed by combining the two protein components individually (8) or using the combined refolded IF proteins in a centrifugal concentration (9) step or other suitable concentration-self-assembly process using an exclusion membrane no greater than 30 kDa with an aqueous buffer containing magnesium or calcium ions or absent metal ions. The IF thread is visualized in the feed or supernatant during concentration and is manually removed (10).

It is expected that the techniques described herein could be used with other IF proteins including other hagfish slime proteins, such as, for example, IF proteins from other hagfish species besides the Eptatretus stoutii proteins used in the below examples.

### Examples

Host organisms genetically engineered to contain intermediate filament proteins (alpha, gamma, or both expressed a continuous single protein chain joined by a flexible linker) are selected for pre-culture. A pre-culture is overgrown overnight in rich-growth media typical for prokaryote or eukaryote protein expression.

Protein expression can be conducted in a bacterial host with a lac operon and antibiotic resistance: 5 mL LB media is pre-cultured overnight then added to 1 L (or more) growth media and grown 3-4 hours until OD₆₀₀=0.5-0.8. The culture is induced with the addition of 0.1 mM-1.0 mM IPTG and grow overnight at 28° C. or for 3 hours at 37° C., while shaking at 250 RPM.

Protein recovery can proceed as follows. Cells are recovered by centrifugation or filtration to isolate the cells away from the growth media. Recovered cells are disrupted by mechanical lysis and/or chemical lysis. Mechanical lysis can include using a French Press and/or microfluidizing technique (high pressure for cell rupture). Typical chemical lysis includes a protease inhibitor, cell lysis enzyme such as lysozyme, and DNAse enzyme to improve sample viscosity (remove nucleic acids) or other suitable chemical lysis method.

Next is the recovery of a soluble protein fraction containing individual intermediate filament proteins from cellular debris: Cell lysate is centrifuged, the soluble protein fraction is decanted as the supernatant and recovered for cobalt or nickel metal-affinity chromatography (IMAC).

In particular, the cell lysate can be centrifuged and the insoluble protein fraction is collected as a pellet and resuspended in buffer containing detergent and low-molarity denaturant (typically 2M urea or guanidine hydrochloride). The insoluble inclusion body is washed in two wash steps: centrifugation, discarding supernatant, resuspended pellet in wash buffer (2M denaturant with detergent in buffer), centrifugation, discarding supernatant, resuspend pellet in wash buffer, centrifugation, discarding supernatant, resuspend remaining pellet in a high concentration of denaturant (8M urea or 6M guanidine, 40 mM imidazole, in aqueous buffer) for IMAC purification using either cobalt or nickel (IMAC) chromatography.

In further embodiments, the cell lysate is centrifuged and the insoluble protein fraction is collected as a pellet, the cell paste is washed with 0.9% NaCl, pelleted, frozen for at least 1 hour, resuspended in buffer for lysis, repelleted after lysis, collect pellet to freeze at least 1 hour, wash pellet two times in aqueous wash buffer containing detergent, pellet by centrifugation, pellet is washed two times in aqueous wash buffer minus detergent, repelleted and stored for purification.

Purification can be one-step IMAC cobalt purification. Protein is loaded onto an IMAC Cobalt column at 1-2 mL/min, washed with 10 column volumes of wash buffer containing at least 40 mM Imidazole or L-Histidine, then eluted with 500 mM imidazole in 2-3 column volumes (CVs). The resulting material is >85% purity.

In further embodiments, the purification can be done by ion exchange chromatography (IEX). Protein is loaded onto an IEX column at 1-2 mL/min, washed with 10 column volumes of wash buffer containing at least 50 mM NaCl, then eluted with maximum 1 M NaCl in 10 column volumes (CVs) in linear or isocratic gradient. The resulting material is >85% purity.

An alternative elution can be performed for a protein construct containing a cleavable His-tag. After washing the column with 10 CV's of wash buffer, the requisite enzyme to cleave the His-tag could be added to elute the intermediate filament proteins. This would enable higher purity material and native material for subsequent refolding and filament assembly.

After purification, the protein(s) remain in high concentration denaturant if the protein material was isolated by inclusion body. For on-column refolding, a step-down or gradient decrease of protein denaturant concentration can be made while the protein remains on the IMAC column. For single protein refolding, the refolding is performed either through step-down denaturant concentrations using dialysis membranes of appropriate molecular weight cutoff to exclude the small organic denaturant and retain the protein or by dilution of the denaturant with the gradual addition of aqueous buffer void of any denaturant.

Dual protein refolding is possible for a two (or more) component intermediate filament system: Since the assembly of the filament typically requires one or more proteins, each protein can be refolding in presence of the second protein to decrease the likelihood of protein aggregation of one protein (self-assembly). In the case of a two protein filament system with one protein more poorly soluble, the two proteins can be refolded together. The one-to-one refolding is performed either through step-down denaturants using dialysis membranes of appropriate molecular weight cutoff to exclude the small organic denaturant and retain the protein or by dilution of the denaturant with the gradual addition of aqueous buffer void of any denaturant.

After complete refolding of the denatured proteins or purification of the native, soluble proteins the assembly of the filaments is completed using one or a combination of the following methods. This method applies to intermediate filament thread formation from proteins refolding as single proteins and mixed at approximately one-to-one ratio, or to the dual protein refolding of two (or more) proteins simultaneously. The following methods promote the close contact of the proteins (one or more) required for the thread formation.

Aqueous buffer exclusion & protein concentration is performed with a porous membrane or filter appropriate to exclude materials of approximately one-half of the molecular weight of the smallest protein component of the filament size or smaller (≤30 kDa molecular weight cutoff for the formation of the filaments from Eptatretus stoutii, which has two proteins of 63 kDa and 67 kDa) exclusion membrane to result in the increased concentration of the individual components.

Ultracentrifugation can promote fiber assembly during sedimentation via centrifugation. The high speed centrifugation overcomes the diffusion and buoyancy to promote one-to-one interactions due to the higher affinity between the two proteins compared to the aggregation induced in each separate protein component. The affinity for the filament and thread formation is higher than the self-assembly.

Small diameter pore assembly by forcing one or more of the required protein components in an inlet through a tube with an inner diameter of less than 1 mm through to an outlet driven by mechanical flow pump or vacuum, gravity, or electroosmotic flow to force the protein solution in to a small, confined space to promote filament assembly and recovery the thread portion at the tube outlet.

Proteinaceous precipitation can be used to exclude water and promote protein interactions according to the Hofmeister series, either by titration of cation-anion salts that induce protein precipitation and collecting the resulting filament filaments that forms or increasing the protein precipitation to form thread filaments during dialysis using salts of the Hofmeister series. The large threads formed can be seen by the naked-eye for easy collection from the solution.

Organic solvent extraction from aqueous solution to increase protein interactions in minimal volumes. Polar, water-immiscible organic solvents such as alcohols, ketones, ethers, carboxylic acids, alkanes, alkenes, alkynes, and halogenated derivatives thereof that promote thread formation without denaturing the intermediate filament proteins.

Filaments obtained according to one or more of these techniques were observed under scanning electron microscopy and found to have appropriate structure.

An example of an Eptatretus stoutii FIL-Alpha DNA sequence (SEQ ID No: 1) is as follows:

| | |
|---|---|
| ATGAGCATCA GCCAGACCGT TAGCAAATCT TACACCAAGT CCGTATCTCG TGGCGGCCAA | 60 |
| GGTGTTAGCT ACAGCCAGAG CAGCAGCCAC AAGGTCGGTG GTGGCAGCGT CCGCTATGGC | 120 |
| ACGACCTATA GCTCCGGCGG TATCAGCCGT GTTCTGGGCT TCCAGGGTGG TGCCGGTGGT | 180 |
| GCTGCAAGCG CGGGTTTTGG CGGTTCGGTT GGCGGTTCCG GTTTGTCACG TGTCCTGGGT | 240 |
| GGCAGCATGG TGAGCGGTTA TCGTAGCGGT ATGGGCGTGG GTGGTCTGAG CCTGAGCGGT | 300 |
| ACGGCAGGCT TGCCGGTGTC TCTGCGTGGC GTGGGTGCTG GTAAAGCACT GCATGCCATT | 360 |
| ACGAGCGCCT TCCGTACCCG TGTTGGTGGC CCTGGCACGT CTGTGGGTGG TTACGGCGTG | 420 |
| AATTACAGCT TCCTGCCAAG CACCGCAGGC CCGTCATTTG GTGGCCCGTT TGGTGGTCCG | 480 |
| TTTGGCGGCC CATTCGGTGG TCCTCTGGGC CCAGGTTACA TCGATCCGGC AACCTTGCCG | 540 |
| TCGCCGGATA CCGTGCAACA TACTCGTATC CGTGAGAAGC AGGATCTGCA AACCCTGAAT | 600 |
| ACCAAATTCG CCAACCTGGT TGATCAAGTG CGCACCCTGG AGCAGCACAA CGCCATTCTG | 660 |
| AAAGCGCAGA TTTCCATGAT TACCAGCCCG TCCGACACTC CGGAAGGCCC GGTCAACACC | 720 |
| GCAGTGGTGG CGAGCACGGT CACCGCCACC TACAACGCGC AAATTGAGGA CTTGCGTACC | 780 |
| ACGAACACGG CCCTGCACAG CGAAATTGAC CACCTGACTA CCATCATTAA TGACATTACG | 840 |
| ACGAAATATG AGGAGCAAGT GGAAGTCACC CGTACGCTGG AAACGGACTG GAATACCAAC | 900 |
| AAAGATAACA TCGATAACAC CTACCTGACC ATTGTGGACT TGCAGACCAA AGTGCAAGGC | 960 |
| CTGGACGAAC AAATCAACAC CACCAAGCAA ATCTATAATG CGCGCGTTCG TGAGGTGCAG | 1020 |
| GCAGCGGTTA CGGGTGGTCC GACTGCGGCC TATAGCATTC GTGTGGACAA TACGCATCAA | 1080 |
| GCGATCGACC TGACGACCTC TCTGCAGGAA ATGAAAACCC ATTATGAAGT TCTGGCAACG | 1140 |
| AAAAGCCGCG AAGAGGCATT TACTCAAGTC CAACCGCGTA TCCAGGAGAT GGCAGTCACG | 1200 |
| GTCCAGGCTG GTCCGCAAGC GATTATCCAA GCGAAAGAGC AGATTCATGT GTTCAAGCTG | 1260 |
| CAAATCGATA GCGTTCACCG TGAAATTGAC CGTCTGCATC GCAAGAATAC CGACGTTGAA | 1320 |
| CGTGAGATTA CGGTGATTGA GACTAATATC CATACCCAGT CCGACGAGTG GACCAATAAC | 1380 |
| ATTAACAGCC TGAAAGTCGA CCTGGAGGTC ATCAAGAAGC AGATTACGCA GTACGCGCGT | 1440 |
| GACTACCAGG ATCTGTTGGC GACGAAAATG TCCCTGGATG TCGAGATCGC AGCGTACAAG | 1500 |
| AAACTGCTGG ATAGCGAAGA AACCCGTATC AGCCACGGTG GCGGTATCAC TATCACCACC | 1560 |
| AACGCGGGTA CCTTCCCGGG TGGTTTGAGC GCTGCACCAG GTGGTGGCGC CAGCTACGCG | 1620 |
| ATGGTCCCTG CTGGCGTCGG TGGTGTTGGC CTGGCGGGTG TTGGCGGTTA CGGCTTTCGT | 1680 |
| AGCATGGGTG GTGGTGGCGG TGTGGGCTAT GGTGCGGGTG GTGGCGGTGT TGGCTATGGT | 1740 |
| GTCGGTGGCG GCTTTGGTGG CGGCATGGGC ATGTCTATGA GCCGCATGAG CATGGGTGCA | 1800 |
| GCAGTGGGCG GTGGTAGCTA CGGCAGCGGT AGCGGTTACT CGGGTGGTTT TGGTTTGTCC | 1860 |
| AGCTCTCGCG CTGGCTACAG CGCGTCCCGT AAGAGCTATA GCAGCGCCCG TAGCAGCAGC | 1920 |
| CGCATCTACC ACCACCATCA CCATCAC | |

An example of an Eptatretus stoutii FIL-Alpha amino sequence including a polyhistidine tag (SEQ ID No: 2) is as follows:

| | |
|---|---|
| MSISQTVSKSYTKSVSRGGQGVSYSQSSSHKVGGGSVRYGTTYSSGGISRVLGFQGGAGG | 60 |
| AASAGFGGSVGGSGLSRVLGGSMVSGYRSGMGVGGLSLSGTAGLPVSLRGVGAGKALHAI | 120 |
| TSAFRTRVGGPGTSVGGYGVNYSFLPSTAGPSFGGPFGGPFGGPFGGPLGPGYIDPATLP | 180 |
| SPDTVQHTRIREKQDLQTLNTKFANLVDQVRTLEQHNAILKAQISMITSPSDTPEGPVNT | 240 |
| AVVASTVTATYNAQIEDLRTTNTALHSELDHLTTIINDITTKYEEQVEVTRTLETDWNTN | 300 |
| KDNIDNTYLTIVDLQTKVQGLDEQINTTKQIYNARVREVQAAVTGGPTAAYSIRVDNTHQ | 360 |
| AIDLTTSLQEMKTHYEVLATKSREEAFTQVQPRIQEMAVTVQAGPQAIIQAKEQIHVFKL | 420 |
| QIDSVHREIDRLHRKNTDVEREITVIETNIHTQSDEWTNNINSLKVDLEVIKKQITQYAR | 480 |
| DYQDLLATKMSLDVEIAAYKKLLDSEETRISHGGGITITTNAGTFPGGLSAAPGGGASYA | 540 |
| MVPAGVGGVGLAGVGGYGFRSMGGGGGVGYGAGGGGVGYGVGGGFGGGMGMSMSRMSMGA | 600 |
| AVGGGSYGSGSGYSGGFGLSSSRAGYSASRKSYSSARSSSRIYHHHHHH | 649 |

An example of an Eptatretus stoutii FIL-Gamma DNA sequence (SEQ ID No: 3) is as follows:

| | |
|---|---|
| ATGGCATCGC ACTCGTCTGT TAGCTATCGT TCCGTTCGCA CTGGTGGCAC CTCCGCAATG | 60 |
| ATCGGTTCTA GCGGTTATGG TGGCAGCTCC AGCTCTCGTG CAATGGGCCT GGGTATGGGT | 120 |
| GCGGCTGGTT TGAGCATGGG CGGTGGTAGC TTTCGTGTGG GCAGCGCTGG CATTGGCGGT | 180 |
| ATGGGCATCA GCTCCGGCAT CGGTGGCATG GGTATTAGCT CACGTGCTGG CGGCATGAGC | 240 |
| GCATACGGCG GTGCGGCTTC TGGTGGTGCA GGCGGTTTCG TGAGCGGTGG CGTTCCAATG | 300 |
| CTGGGTTACG GTGGTGGTGC GGGTGGCTTT ATCGGTGGTG TGAGCCCAGG CATCATGGCG | 360 |
| AGCCCGGCAT TTACTGCTGG TCGTGCAATT ACCAGCGCGG GTATGAGCGG CGTTGTTGGC | 420 |
| ACGTTGGGTC CTGCCGGTGG TATGGTGCCG AGCCTGGTGA GCCGTGACGA GGTCAAAAAC | 480 |
| ATCCTGGGCA CGCTGAATCA ACGTCTGGCG AGCTATGTGG ACAAAGTCCG CCAGCTGACG | 540 |
| ATCGAGAATG AGACTATGGA AGAGGAGCTG AAGAACCTGA CTGGCGGCGT TCCGATGAGC | 600 |
| CCGGATAGCA CCGTCAACCT GGAAAACGTT GAGACTCAAG TCACCGAGAT GCTGACCGAA | 660 |
| GTGAGCAACC TGACCTTGGA GCGCGTTCGT CTGGAGATTG ATGTTGACCA CTTGCGTGCG | 720 |
| ACGGCAGATG AAATCAAGTC CAAATACGAA TTCGAACTGG GTGTGCGTAT GCAATTGGAA | 780 |
| ACGGATATTG CCAATATGAA GCGTGATCTT GAAGCGGCCA ATGATATGCG CGTCGACCTG | 840 |
| GATAGCAAAT TCAACTTCCT GACGGAGGAG CTGACCTTCC AGCGTAAAAC GCAGATGGAA | 900 |
| GAACTGAATA CCCTGAAGCA GCAATTCGGT CGTCTGGGTC CGGTGCAGAC GTCCGTGATT | 960 |
| GAACTGGATA ATGTGAAATC CGTGAATCTG ACGGATGCCC TGAACGTTAT GCGCGAGGAG | 1020 |
| TATCAGCAAG TTGTGACGAA AAACGTCCAA GAAGCCGAAA CCTATTGTAA AATGCAGATT | 1080 |
| GACCAGATCC AAGGTATCTC GACCCAAACC ACCGAGCAGA TTAGCATCCT GGACAAGGAA | 1140 |
| ATCAATACGC TGGAGAAGGA GCTGCAGCCG CTGAACGTCG AGTACCAGCG CCTGCTGACC | 1200 |
| ACCTATCAGA CCCTGGGCGA CCGTCTGACC GATCTGCAGA ATCGTGAAAG CATTGACCTG | 1260 |
| GTGCAATTTC AAAATACCTA CACCCGTTAC GAGCAAGAGA TTGAAGGCAA TCAAGTTGAC | 1320 |
| TTGCAGCGCC AACTGGTGAC CTATCAGCAA CTGCTCGACG TTAAAACGGC ATTGGACGCG | 1380 |
| GAAATCGCGA CCTACAAGAA ACTGCTGGAA GGCCAAGAGT TGATGGTCCG CACTGCAATG | 1440 |
| GCCGATGATT TTGCCCATGC TACTGTCGTT CGTAGCGGTA CCCTGGGTGG CGCAAGCAGC | 1500 |
| AGCTCTGTCG GCTATGGCGC GTCTAGCACC ACGCTGGGTG CGATCAGCGG TGGCTACAGC | 1560 |
| ACCGGTGGCG GTGCAAGCTA CTCTGCTGGT GCCGGTGGTG CCAGCTATTC CGCTGGTGCG | 1620 |
| GGTGGTGCTT CATACGGTGT TGGTGGCGGT TATAGCGGCG GTAGCTCTGC GATGATGGAG | 1680 |
| GGTAGCAGCA GCGGCGGTCA CAGCATGTAC AGCAGCAGCA GCATGAAGCG TAGCTCCTCC | 1740 |
| AAGTCCGCGT CTGCAAGCGC GGGTGGTTAC GGCACCAGCG GTCATGACTC CACCATTATT | 1800 |
| CTGCAGCAGC ACCACCATCA TCACCAC | |

An example of an Eptatretus stoutii FIL-Gamma amino sequence including a polyhistidine tag (SEQ ID No: 2) is as follows:

| | |
|---|---|
| MASHSSVSYRSVRTGGTSAMIGSSGYGGSSSSRAMGLGMGAAGLSMGGGSFRVGSAGIGG | 60 |
| MGISSGIGGMGISSRAGGMSAYGGAASGGAGGFVSGGVPMLGYGGGAGGFIGGVSPGIMA | 120 |
| SPAFTAGRAITSAGMSGVVGTLGPAGGMVPSLVSRDEVKNILGTLNQRLASYVDKVRQLT | 180 |
| IENETMEEELKNLTGGVPMSPDSTVNLENVETQVTEMLTEVSNLTLERVRLEIDVDHLRA | 240 |
| TADEIKSKYEFELGVRMQLETDIANMKRDLEAANDMRVDLDSKFNFLTEELTFQRKTQME | 300 |
| ELNTLKQQFGRLGPVQTSVIELDNVKSVNLTDALNVMREEYQQVVTKNVQEAETYCKMQI | 360 |
| DQIQGISTQTTEQISILDKEINTLEKELQPLNVEYQRLLTTYQTLGDRLTDLQNRESIDL | 420 |
| VQFQNTYTRYEQEIEGNQVDLQRQLVTYQQLLDVKTALDAEIATYKKLLEGQELMVRTAM | 480 |
| ADDFAHATVVRSGTLGGASSSSVGYGASSTTLGAISGGYSTGGGASYSAGAGGASYSAGA | 540 |
| GGASYGVGGGYSGGSSAMMEGSSSGGHSMYSSSSMKRSSSKSASASAGGYGTSGHDSTII | 600 |
| LQQHHHHHH | 609 |

Presence of the polyhistidine tag did not impair filament assembly. Nonetheless, it is possible to introduce a cleavage site to create a cleavable tag than can be removed during or after purification.

An example of a polyhistidine tagged combined protein with Alpha(spacer)Gamma (both proteins in N-terminal to C-terminal direction) is as follows: (SEQ ID No: 5)

(His)ₙMSISQTVSKSYTKSVSRGGQGVSYSQSSSHKVGGGSVRYGTTY
SSGGISRVLGFQGGAGGAASAGFGGSVGGSGLSRVLGGSMVSGYRSGM
GVGGLSLSGTAGLPVSLRGVGAGKALHAITSAFRTRVGGPGTSVGGYGV
NYSFLPSTAGPSFGGPFGGPFGGPFGGPLGPGYIDPATLPSPDTVQHTR
IREKQDLQTLNTKFANLVDQVRTLEQHNAILKAQISMITSPSDTPEGPV
NTAVVASTVTATYNAQIEDLRTTNTALHSEIDHLTTIINDITTKYEEQV
EVTRTLETDWNTNKDNIDNTYLTIVDLQTKVQGLDEQINTTKQIYNARV
REVQAAVTGGPTAAYSIRVDNTHQAIDLTTSLQEMKTHYEVLATKSREE
AFTQVQPRIQEMAVTVQAGPQATIQAKEQIHVEKLQIDSVHREIDRLHR
KNTDVEREITVIETNIHTQSDEWTNNINSLKVDLEVIKKQITQYARDYQ
DLLATKMSLDVEIAAYKKLLDSEETRISHGGGITITTNAGTEPGGLSAA
PGGGASYAMVPAGVGGVGLAGVGGYGFRSMGGGGGVGYGAGGGGVGYGV
GGGFGGGMGMSMSRMSMGAAVGGGSYGSGSGYSGGFGLSSSRAGYSASR
KSYSSARSSSRIY(Gly)ₙ(Ser)ₙ(Gly)n(Ser)ₙMASHSSVSYRSVR
TGGTSAMIGSSGYGGSSSSRAMGLGMGAAGLSMGGGSFRVGSAGIGGMG
ISSGIGGMGISSRAGGMSAYGGAASGGAGGEVSGGVPMLGYGGGAGGFI
GGVSPGIMASPAFTAGRAITSAGMSGVVGTLGPAGGMVPSLVSRDEVKN
ILGTLNQRLASYVDKVRQLTIENETMEEELKNLTGGVPMSPDSTVNLEN
VETQVTEMLTEVSNLTLERVRLEIDVDHLRATADEIKSKYEFELGVRMQ
LETDIANMKRDLEAANDMRVDLDSKFNFLTEELTFQRKTQMEELNTLKQ
QFGRLGPVQTSVIELDNVKSVNLTDALNVMREEYQQVVTKNVQEAETYC
KMQIDQIQGISTQTTEQISILDKEINTLEKELQPLNVEYQRLLTTYQTL
GDRLTDLQNRESIDLVQFQNTYTRYEQEIEGNQVDLQRQLVTYQQLLDV
KTALDAEIATYKKLLEGQELMVRTAMADDFAHATVVRSGTLGGASSSSV
GYGASSTTLGAISGGYSTGGGASYSAGAGGASYSAGAGGASYGVGGGYS
GGSSAMMEGSSSGGHSMYSSSSMKRSSSKSASASAGGYGTSGHDSTIIL
QQ(His)ₙ

where n=0 to 10 repeats. One of ordinary skill in the art may modify the spacer sequence by altering its length and composition. The location of the histidine tags may also be varied.

An example of a polyhistidine tagged combined protein of Alpha(spacer)Gamma with Alpha in the N-terminal to C-terminal direction; and Gamma in C-terminal to N-terminal sequence direction is as follows (SEQ ID No: 6):

(His)ₙMSISQTVSKSYTKSVSRGGQGVSYSQSSSHKVGGGSVRYGTTY
SSGGISRVLGFQGGAGGAASAGFGGSVGGSGLSRVLGGSMVSGYRSGM
GVGGLSLSGTAGLPVSLRGVGAGKALHAITSAFRTRVGGPGTSVGGYGV
NYSFLPSTAGPSFGGPFGGPFGGPFGGPLGPGYIDPATLPSPDTVQHTR
IREKQDLQTLNTKFANLVDQVRTLEQHNAILKAQISMITSPSDTPEGPV
NTAVVASTVTATYNAQIEDLRTTNTALHSEIDHLTTIINDITTKYEEQV
EVTRTLETDWNTNKDNIDNTYLTIVDLQTKVQGLDEQINTTKQIYNARV
REVQAAVTGGPTAAYSIRVDNTHQAIDLTTSLQEMKTHYEVLATKSREE
AFTQVQPRIQEMAVTVQAGPQATIQAKEQIHVFKLQIDSVHREIDRLHR
KNTDVEREITVIETNIHTQSDEWTNNINSLKVDLEVIKKQITQYARDYQ
DLLATKMSLDVEIAAYKKLLDSEETRISHGGGITITTNAGTFPGGLSAA
PGGGASYAMVPAGVGGVGLAGVGGYGFRSMGGGGGVGYGAGGGGVGYGV
GGGFGGGMGMSMSRMSMGAAVGGGSYGSGSGYSGGFGLSSSRAGYSASR
KSYSSARSSSRIY(Gly)ₙ(Ser)ₙ(Gly)ₙ(Ser)ₙQQLIITSDHGST
GYGGASASASKSSSRKMSSSSYMSHGGSSSGEMMASSGGSYGGGVGYSA
GGAGASYSAGGAGASYSAGGGTSYGGSIAGLTTSSAGYGVSSSSAGGLT
GSRVVTAHAFDDAMATRVMLEQGELLKKYTAIEADLATKVDLLQQYTVL
QRQLDVQNGEIEQEYRTYTNQFQVLDISERNQLDTLRDGLTQYTTLLRQ
YEVNLPQLEKELTNIEKDLISIQETTQTSIGQIQDIQMKCYTEAEQVNK
TVVQQYEERMVNLADTLNVSKVNDLEIVSTQVPGLRGFQQKLTNLEEMQ
TKRQFTLEETLFNFKSDLDVRMDNAAELDRKMNAIDTELQMRVGLEFEY
KSKIEDATARLHDVDIERELTLNSVETLMETVQTEVNELNVTSDPSMPV
GGTLNKLEEEMTENEITLQRVKDVYSALRQNLTGLINKVEDRSVLSPVM
GGAPGLTGVVGSMGASTIARGATFAPSAMIGPSVGGIFGGAGGGYGLMP
VGGSVFGGAGGSAAGGYASMGGARSSIGMGGIGSSIGMGGIGASGVRFS
GGGMSLGAAGMGLGMARSSSSGGYGSSGIMASTGGTRVSRYSVSSHSAM
(His)ₙ

where n=0 to 10 repeats. One of ordinary skill in the art may modify the spacer sequence by altering its length and composition. The location of the histidine tags may also be varied.

Applications

The recombinant IF proteins are expected to find use in a number of areas. It is expected that might be applied as anti-fouling coatings for marine vessels and equipment, drag reduction material, to counter autonomous and unmanned applications. Other possible uses include wound dressings including burns, as diver anti-shark spray, biosensors and bioelectronics, firefighting material, ballistic protection material, chemical and biological cleanup material, high-strength additive for polymers, and textile materials.

Advantages

The native, untagged IF protein in mild denaturing conditions in alkaline aqueous buffer is amenable to large batch growth (scale-up) and purification using non-IMAC purification. Further advantages of the mild denaturing, alkaline conditions for IF protein formation include more rapidly refolding the IF proteins by denaturant removal for faster manufacturing of IF's, and may be suitable for batch for fermentation, bioreactor, and batch processing.

The described technique for polyHis.tag recombinant IF protein systems includes purification under denaturing conditions, which is advantageous for isolating the IF proteins from inclusion bodies during bacterial growth and lysis. The polyHis.tag enables IMAC purification for one-step high purification of the IF proteins and allows for on column refolding of the IF protein, thereby rapidly decreasing the refolding time using conventional step-down denaturant methods.

The polyHis-tag IF protein is amenable to large batch growth (scale-up) and purification since affinity purification methods are not concentration limited. Further advantages of the polyHis-tag IF protein include appending any divalent metal, include magnetic ions, for a magnetically attachable IF protein and IF thread. The polyHis-tag can be readily attached to nanomaterials and metal surfaces for biosensor and bioelectronics. The polyHis is amenable to quantum dot and semiconductor attachment for fluorescent tracking capabilities.

### CONCLUDING REMARKS

All documents mentioned herein are hereby incorporated by reference for the purpose of disclosing and describing the particular materials and methodologies for which the document was cited.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

### REFERENCES

Jing Fu, Paul A. Guerette, and Ali Miserez; “Self-Assembly of Recombinant Hagfish Thread Keratins Amenable to a Strain-Induced α-Helix to β-Sheet Transition,” Biomacromolecules (2015), 16 (8), pp 2327-2339.
U.S. Pat. No. 7,049,405

What is claimed is:

1. A method of preparing intermediate filament threads, the method comprising:
growing cells of a host organism engineered to produce at least one intermediate filament and causing the cells to produce the at least one intermediate filament protein; then
recovering and lysing the cells to extract the at least one intermediate filament protein; then
purifying the at least one intermediate filament protein under denaturing conditions; then
causing the at least one intermediate filament protein to refold; and then
allowing the at least one intermediate filament protein to form a thread.

2. The method of claim 1, wherein the at least one intermediate filament protein comprises at least one hagfish slime protein.

3. The method of claim 2, wherein the at least one intermediate filament protein comprises SEQ ID No: 1 and/or SEQ ID No: 2.

4. The method of claim 2, wherein the at least one intermediate filament protein comprises SEQ ID No: 5 or SEQ ID No: 6.

5. The method of claim 1, wherein the at least one intermediate filament protein comprises both an alpha protein and a gamma protein expressed together joined by a spacer.

6. The method of claim 1, wherein two different intermediate filament proteins are purified separately and then combined for refolding.

7. The method of claim 1, wherein the at least one intermediate filament protein is purified on a column and the causing the at least one intermediate filament protein to refold is performed on the same column.

8. The method of claim 1, wherein the at least one intermediate filament protein comprises a tag for affinity purification.

9. The method of claim 1, wherein the at least one intermediate filament protein is a native, untagged protein and the denaturing conditions are mild.

10. A method of preparing intermediate filament threads, the method comprising:
growing cells of a host organism engineered to produce at least one intermediate filament and causing the cells to produce the at least one intermediate filament protein, wherein the at least one intermediate filament protein comprises a tag for affinity purification; then
recovering and lysing the cells to extract the at least one intermediate filament protein; then
purifying the at least one intermediate filament protein under denaturing conditions on an affinity column; then
causing the at least one intermediate filament protein to refold on the affinity column; and then
allowing the at least one intermediate filament protein for to form a thread.

11. The method of claim 10, wherein the at least one intermediate filament protein comprises at least one hagfish slime protein.

12. The method of claim 11, wherein the at least one intermediate filament protein comprises SEQ ID No: 1 and/or SEQ ID No: 2.

13. The method of claim 11, wherein the at least one intermediate filament protein comprises SEQ ID No: 5 or SEQ ID No: 6.

14. The method of claim 10, wherein the at least one intermediate filament protein comprises both an alpha protein and a gamma protein expressed together joined by a spacer.

15. The method of claim 10, wherein two different intermediate filament proteins are purified separately and then combined for refolding.


SEQUENCE LISTING (6 sequences)

SEQ ID NO: 1 - DNA, 1947 bp, Eptatretus stouti
atgagcatcagccagaccgttagcaaatcttacaccaagtccgtatctcgtggcggccaaggtgttagctacagccagagcagcagccacaaggtcggtggtggcagcgtccgctatggcacgacctatagctccggcggtatcagccgtgttctgggcttccagggtggtgccggtggtgctgcaagcgcgggttttggcggttcggttggcggttccggtttgtcacgtgtcctgggtggcagcatggtgagcggttatcgtagcggtatgggcgtgggtggtctgagcctgagcggtacggcaggcttgccggtgtctctgcgtggcgtgggtgctggtaaagcactgcatgccattacgagcgccttccgtacccgtgttggtggccctggcacgtctgtgggtggttacggcgtgaattacagcttcctgccaagcaccgcaggcccgtcatttggtggcccgtttggtggtccgtttggcggcccattcggtggtcctctgggcccaggttacatcgatccggcaaccttgccgtcgccggataccgtgcaacatactcgtatccgtgagaagcaggatctgcaaaccctgaataccaaattcgccaacctggttgatcaagtgcgcaccctggagcagcacaacgccattctgaaagcgcagatttccatgattaccagcccgtccgacactccggaaggcccggtcaacaccgcagtggtggcgagcacggtcaccgccacctacaacgcgcaaattgaggacttgcgtaccacgaacacggccctgcacagcgaaattgaccacctgactaccatcattaatgacattacgacgaaatatgaggagcaagtggaagtcacccgtacgctggaaacggactggaataccaacaaagataacatcgataacacctacctgaccattgtggacttgcagaccaaagtgcaaggcctggacgaacaaatcaacaccaccaagcaaatctataatgcgcgcgttcgtgaggtgcaggcagcggttacgggtggtccgactgcggcctatagcattcgtgtggacaatacgcatcaagcgatcgacctgacgacctctctgcaggaaatgaaaacccattatgaagttctggcaacgaaaagccgcgaagaggcatttactcaagtccaaccgcgtatccaggagatggcagtcacggtccaggctggtccgcaagcgattatccaagcgaaagagcagattcatgtgttcaagctgcaaatcgatagcgttcaccgtgaaattgaccgtctgcatcgcaagaataccgacgttgaacgtgagattacggtgattgagactaatatccatacccagtccgacgagtggaccaataacattaacagcctgaaagtcgacctggaggtcatcaagaagcagattacgcagtacgcgcgtgactaccaggatctgttggcgacgaaaatgtccctggatgtcgagatcgcagcgtacaagaaactgctggatagcgaagaaacccgtatcagccacggtggcggtatcactatcaccaccaacgcgggtaccttcccgggtggtttgagcgctgcaccaggtggtggcgccagctacgcgatggtccctgctggcgtcggtggtgttggcctggcgggtgttggcggttacggctttcgtagcatgggtggtggtggcggtgtgggctatggtgcgggtggtggcggtgttggctatggtgtcggtggcggctttggtggcggcatgggcatgtctatgagccgcatgagcatgggtgcagcagtgggcggtggtagctacggcagcggtagcggttactcgggtggttttggtttgtccagctctcgcgctggctacagcgcgtcccgtaagagctatagcagcgcccgtagcagcagccgcatctaccaccaccatcaccatcac

SEQ ID NO: 2 - protein, 649 aa, Eptatretus stouti
MSISQTVSKSYTKSVSRGGQGVSYSQSSSHKVGGGSVRYGTTYSSGGISRVLGFQGGAGGAASAGFGGSVGGSGLSRVLGGSMVSGYRSGMGVGGLSLSGTAGLPVSLRGVGAGKALHAITSAFRTRVGGPGTSVGGYGVNYSFLPSTAGPSFGGPFGGPFGGPFGGPLGPGYIDPATLPSPDTVQHTRIREKQDLQTLNTKFANLVDQVRTLEQHNAILKAQISMITSPSDTPEGPVNTAVVASTVTATYNAQIEDLRTTNTALHSEIDHLTTIINDITTKYEEQVEVTRTLETDWNTNKDNIDNTYLTIVDLQTKVQGLDEQINTTKQIYNARVREVQAAVTGGPTAAYSIRVDNTHQAIDLTTSLQEMKTHYEVLATKSREEAFTQVQPRIQEMAVTVQAGPQAIIQAKEQIHVFKLQIDSVHREIDRLHRKNTDVEREITVIETNIHTQSDEWTNNINSLKVDLEVIKKQITQYARDYQDLLATKMSLDVEIAAYKKLLDSEETRISHGGGITITTNAGTFPGGLSAAPGGGASYAMVPAGVGGVGLAGVGGYGFRSMGGGGGVGYGAGGGGVGYGVGGGFGGGMGMSMSRMSMGAAVGGGSYGSGSGYSGGFGLSSSRAGYSASRKSYSSARSSSRIYHHHHHH

SEQ ID NO: 3 - DNA, 1827 bp, Eptatretus stouti
atggcatcgcactcgtctgttagctatcgttccgttcgcactggtggcacctccgcaatgatcggttctagcggttatggtggcagctccagctctcgtgcaatgggcctgggtatgggtgcggctggtttgagcatgggcggtggtagctttcgtgtgggcagcgctggcattggcggtatgggcatcagctccggcatcggtggcatgggtattagctcacgtgctggcggcatgagcgcatacggcggtgcggcttctggtggtgcaggcggtttcgtgagcggtggcgttccaatgctgggttacggtggtggtgcgggtggctttatcggtggtgtgagcccaggcatcatggcgagcccggcatttactgctggtcgtgcaattaccagcgcgggtatgagcggcgttgttggcacgttgggtcctgccggtggtatggtgccgagcctggtgagccgtgacgaggtcaaaaacatcctgggcacgctgaatcaacgtctggcgagctatgtggacaaagtccgccagctgacgatcgagaatgagactatggaagaggagctgaagaacctgactggcggcgttccgatgagcccggatagcaccgtcaacctggaaaacgttgagactcaagtcaccgagatgctgaccgaagtgagcaacctgaccttggagcgcgttcgtctggagattgatgttgaccacttgcgtgcgacggcagatgaaatcaagtccaaatacgaattcgaactgggtgtgcgtatgcaattggaaacggatattgccaatatgaagcgtgatcttgaagcggccaatgatatgcgcgtcgacctggatagcaaattcaacttcctgacggaggagctgaccttccagcgtaaaacgcagatggaagaactgaataccctgaagcagcaattcggtcgtctgggtccggtgcagacgtccgtgattgaactggataatgtgaaatccgtgaatctgacggatgccctgaacgttatgcgcgaggagtatcagcaagttgtgacgaaaaacgtccaagaagccgaaacctattgtaaaatgcagattgaccagatccaaggtatctcgacccaaaccaccgagcagattagcatcctggacaaggaaatcaatacgctggagaaggagctgcagccgctgaacgtcgagtaccagcgcctgctgaccacctatcagaccctgggcgaccgtctgaccgatctgcagaatcgtgaaagcattgacctggtgcaatttcaaaatacctacacccgttacgagcaagagattgaaggcaatcaagttgacttgcagcgccaactggtgacctatcagcaactgctcgacgttaaaacggcattggacgcggaaatcgcgacctacaagaaactgctggaaggccaagagttgatggtccgcactgcaatggccgatgattttgcccatgctactgtcgttcgtagcggtaccctgggtggcgcaagcagcagctctgtcggctatggcgcgtctagcaccacgctgggtgcgatcagcggtggctacagcaccggtggcggtgcaagctactctgctggtgccggtggtgccagctattccgctggtgcgggtggtgcttcatacggtgttggtggcggttatagcggcggtagctctgcgatgatggagggtagcagcagcggcggtcacagcatgtacagcagcagcagcatgaagcgtagctcctccaagtccgcgtctgcaagcgcgggtggttacggcaccagcggtcatgactccaccattattctgcagcagcaccaccatcatcaccac

SEQ ID NO: 4 - protein, 489 aa, Eptatretus stouti
SPAFTAGRAITSAGMSGVVGTLGPAGGMVPSLVSRDEVKNILGTLNQRLASYVDKVRQLTIENETMEEELKNLTGGVPMSPDSTVNLENVETQVTEMLTEVSNLTLERVRLEIDVDHLRATADEIKSKYEFELGVRMQLETDIANMKRDLEAANDMRVDLDSKFNFLTEELTFQRKTQMEELNTLKQQFGRLGPVQTSVIELDNVKSVNLTDALNVMREEYQQVVTKNVQEAETYCKMQIDQIQGISTQTTEQISILDKEINTLEKELQPLNVEYQRLLTTYQTLGDRLTDLQNRESIDLVQFQNTYTRYEQEIEGNQVDLQRQLVTYQQLLDVKTALDAEIATYKKLLEGQELMVRTAMADDFAHATVVRSGTLGGASSSSVGYGASSTTLGAISGGYSTGGGASYSAGAGGASYSAGAGGASYGVGGGYSGGSSAMMEGSSSGGHSMYSSSSMKRSSSKSASASAGGYGTSGHDSTIILQQHHHHHH

SEQ ID NO: 5 - protein, 1252 aa, Artificial Sequence
  synthetic construct
  REPEAT (1)..(1): 1- 10 repeats
  REPEAT (645)..(645): 1- 10 repeats
  REPEAT (646)..(646): 1- 10 repeats
  REPEAT (647)..(647): 1- 10 repeats
  REPEAT (648)..(648): 1- 10 repeats
  REPEAT (1252)..(1252): 1- 10 repeats
HMSISQTVSKSYTKSVSRGGQGVSYSQSSSHKVGGGSVRYGTTYSSGGISRVLGFQGGAGGAASAGFGGSVGGSGLSRVLGGSMVSGYRSGMGVGGLSLSGTAGLPVSLRGVGAGKALHAITSAFRTRVGGPGTSVGGYGVNYSFLPSTAGPSFGGPFGGPFGGPFGGPLGPGYIDPATLPSPDTVQHTRIREKQDLQTLNTKFANLVDQVRTLEQHNAILKAQISMITSPSDTPEGPVNTAVVASTVTATYNAQIEDLRTTNTALHSEIDHLTTIINDITTKYEEQVEVTRTLETDWNTNKDNIDNTYLTIVDLQTKVQGLDEQINTTKQIYNARVREVQAAVTGGPTAAYSIRVDNTHQAIDLTTSLQEMKTHYEVLATKSREEAFTQVQPRIQEMAVTVQAGPQAIIQAKEQIHVFKLQIDSVHREIDRLHRKNTDVEREITVIETNIHTQSDEWTNNINSLKVDLEVIKKQITQYARDYQDLLATKMSLDVEIAAYKKLLDSEETRISHGGGITITTNAGTFPGGLSAAPGGGASYAMVPAGVGGVGLAGVGGYGFRSMGGGGGVGYGAGGGGVGYGVGGGFGGGMGMSMSRMSMGAAVGGGSYGSGSGYSGGFGLSSSRAGYSASRKSYSSARSSSRIYGSGSMASHSSVSYRSVRTGGTSAMIGSSGYGGSSSSRAMGLGMGAAGLSMGGGSFRVGSAGIGGMGISSGIGGMGISSRAGGMSAYGGAASGGAGGFVSGGVPMLGYGGGAGGFIGGVSPGIMASPAFTAGRAITSAGMSGVVGTLGPAGGMVPSLVSRDEVKNILGTLNQRLASYVDKVRQLTIENETMEEELKNLTGGVPMSPDSTVNLENVETQVTEMLTEVSNLTLERVRLEIDVDHLRATADEIKSKYEFELGVRMQLETDIANMKRDLEAANDMRVDLDSKFNFLTEELTFQRKTQMEELNTLKQQFGRLGPVQTSVIELDNVKSVNLTDALNVMREEYQQVVTKNVQEAETYCKMQIDQIQGISTQTTEQISILDKEINTLEKELQPLNVEYQRLLTTYQTLGDRLTDLQNRESIDLVQFQNTYTRYEQEIEGNQVDLQRQLVTYQQLLDVKTALDAEIATYKKLLEGQELMVRTAMADDFAHATVVRSGTLGGASSSSVGYGASSTTLGAISGGYSTGGGASYSAGAGGASYSAGAGGASYGVGGGYSGGSSAMMEGSSSGGHSMYSSSSMKRSSSKSASASAGGYGTSGHDSTIILQQH

SEQ ID NO: 6 - protein, 1252 aa, Artificial Sequence
  synthetic construct
  REPEAT (1)..(1): 1 -10 repeats
  REPEAT (809)..(809): 1 -10 repeats
  REPEAT (810)..(810): 1 -10 repeats
  REPEAT (811)..(811): 1 -10 repeats
  REPEAT (812)..(812): 1 -10 repeats
  REPEAT (1252)..(1252): 1 -10 repeats
HMSISQTVSKSYTKSVSRGGQGVSYSQSSSHKVGGGSVRYGTTYSSGGISRVLGFQGGAGGAASAGFGGSVGGSGLSRVLGGSMVSGYRSGMGVGGLSLSGTAGLPVSLRGVGAGKALHAITSAFRTRVGGPGTSVGGYGVNYSFLPSTAGPSFGGPFGGPFGGPFGGPLGPGYIDPATLPSPDTVQHTRIREKQDLQTLNTKFANLVDQVRTLEQHNAILKAQISMITSPSDTPEGPVNTAVVASTVTATYNAQIEDLRTTNTALHSEIDHLTTIINDITTKYEEQVEVTRTLETDWNTNKDNIDNTYLTIVDLQTKVQGLDEQINTTKQIYNARVREVQAAVTGGPTAAYSIRVDNTHQAIDLTTSLQEMKTHYEVLATKSREEAFTQVQPRIQEMAVTVQAGPQAIIQAKEQIHVFKLQIDSVHREIDRLHRKNTDVEREITVIETNIHTQSDEWTNNINSLKVDLEVIKKQITQYARDYQDLLATKMSLDVEIAAYKKLLDSEETRISHGGGITITTNAGTFPGGLSAAPGGGASYAMVPAGVGGVGLAGVGGYGFRSMGGGGGVGYGAGGGGVGYGVGGGFGGGMGMSMSRMSMGAAVGGGSYGSGSGYSGGFGLSSSRAGYSASRKSYSSARSSSRIYGSGSQQLIITSDHGSTGYGGASASASKSSSRKMSSSSYMSHGGSSSGEMMASSGGSYGGGVGYSAGGAGASYSAGGAGASYSAGGGTSYGGSIAGLTTSSAGYGVSSSSAGGLTGSRVVTAHAFDDAMATRVMLEQGELLKKYTAIEADLATKVDLLQQYTVLQRQLDVQNGEIEQEYRTYTNQFQVLDISERNQLDTLRDGLTQYTTLLRQYEVNLPQLEKELTNIEKDLISIQETTQTSIGQIQDIQMKCYTEAEQVNKTVVQQYEERMVNLADTLNVSKVNDLEIVSTQVPGLRGFQQKLTNLEEMQTKRQFTLEETLFNFKSDLDVRMDNAAELDRKMNAIDTELQMRVGLEFEYKSKIEDATARLHDVDIELRVRELTLNSVETLMETVQTEVNELNVTSDPSMPVGGTLNKLEEEMTENEITLQRVKDVYSALRQNLTGLINKVEDRSVLSPVMGGAPGLTGVVGSMGASTIARGATFAPSAMIGPSVGGIFGGAGGGYGLMPVGGSVFGGAGGSAAGGYASMGGARSSIGMGGIGSSIGMGGIGASGVRFSGGGMSLGAAGMGLGMARSSSSGGYGSSGIMASTGGTRVSRYSVSSHSAMH